(19) United States
(12) Patent Application Publication
(10) Pub. No.: US 2009/0093777 A1
(43) Pub. Date: Apr. 9, 2009

(54) Aswipe

(76) Inventor: Robert L. Vilar, Pewaukee, WI (US)

Correspondence Address: ROBERT L. VILAR, 555 C GRANDVIEW CT, PEWAUKEE, WI, 53072, US

(21) Appl. No.: 11/973,996

(22) Filed: Oct. 4, 2007

(51) Int. Cl. A61M 35/00

(52) U.S. Cl. 604/289

(57) ABSTRACT

This invention, Aswipe, is a pad lubricated with a white vasoline like pure petroleum jelly on a non-textile material like Kleenex, Viva paper towel, folded into a pad as shown in drawings. On page 3. FIG. 1, the open sheet of soft material, Viva paper towel size (3″×11″). FIG. 2, the sheet in FIG. 1 is folded in ½ and the center section, shown as a screened pattern (FIG. 3 page 4) is the lubricated area.

(A gentle lubricant, such as Vasoline, pure petroleum jelly).

The size is now (3″×5½″). FIG. 3, the sheet in FIG. 3 is folded in ½ once more to a finished size of (3″×2⅞″). FIG. 5 the finished lubricated pad in FIG. 4, ready for use, is then vacuum packed and heat-sealed into a plastic package, ready for use.

This invention involves applying a vaseline type lubricant to a disposable pad, folding the lubricant into the pad and packaging it for consumer use.

The purpose of this lubricated pad is to apply the jell or ointment to private rectal and/or vaginal parts to prevent chapping and soreness in these areas. The jell is applied after using the toilet and first wiping with regular toilet paper, then the lubricated pad is used. Having this small disposable pad enables consumers to house it either in their bathrooms or carry some in their pockets when they are out and about. It can be applied without getting the jell all over their hands. And the jelled pads can be flushed. The main components of this invention are the lubricant and the disposable tissue-like pad on which the lubricant is placed, plus the packaging. At the moment lubricants are found either in ajar or a tube. This invention enables the lubricant to be more easily carried in a pocket, applied without mess and the pad easily disposed of.

### A BRIEF DESCRIPTION OF THE DRAWING

A swipe as shown in

FIG. 1 consists of a sheet of Kleenex, Viva soft paper towel (3″×11″). This sheet is folded in half to (3½″×5½″), see

FIG. 2. The center section, an area of (1″×3″) is coated with a gentle lubricant such as vaseline, shown in

FIG. 3. Again this sheet is folded in half to (3″×2⅞″) in

FIG. 4. The lubricant is in the center of this folded section of the pad. This completed pad is then vacuum heat-sealed in acetate as shown in

FIG. 5.]

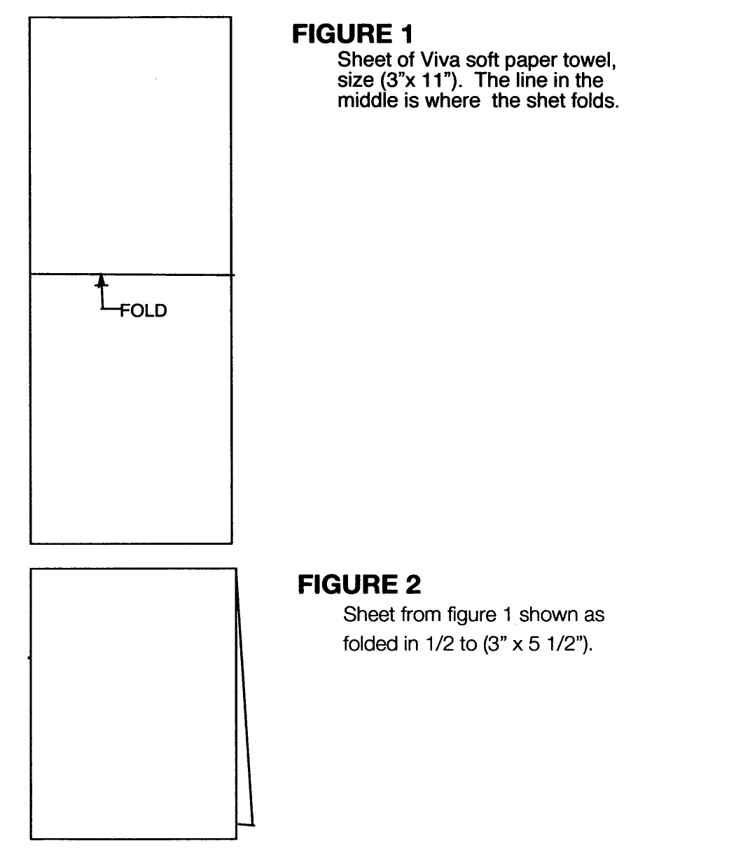

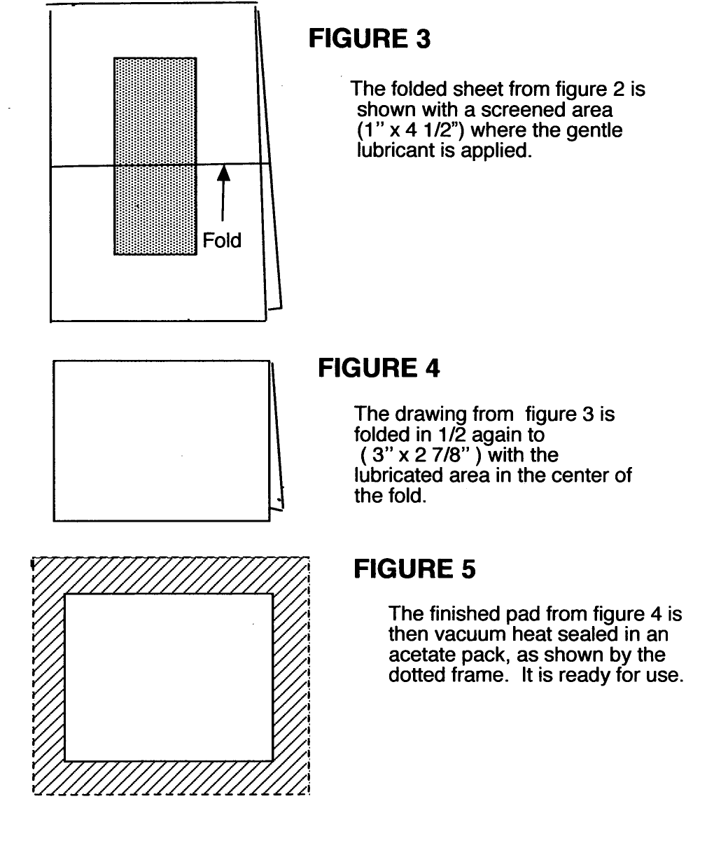

### BACKGROUND

I, Robert L. Vilar, got the idea for this invention because I found that while lubricating petroleum jelly and ointments at the moment come in jars and tubes, applying the lubricant from these containers is inconvenient to carry around and messy to use. Having the lubricant on an individual tissue-like pad is convenient to pocket and carry about and does not gum up a person's hands when using it. This is a much improved method of applying petroleum jelly and/or ointment to a person's private rectal and/or vaginal areas.

1. I Robert l. Vilar, claim all rights of ownership: to the Aswipe product that I invented and want to develop and produce. It is a petrolum jelly substance placed on a diposable tissue-like pad that is folded to size (3″ by 2⅞″) and placed into a small vacuum sealed package, that can be kept in a bathroom or easily carried about in a pocket to be opened and used as a gentle lubricant on private rectal and vaginal parts to aid in chapping or soreness in these areas after using the toilet paper and enabling the consumer to apply the jelly-like subsance without getting it all over his/her hands.
This claim is limited to its use as a lubricant. It is not antibacterial or medicated.
